(19) United States
(12) Patent Application Publication
(10) Pub. No.: US 2010/0215700 A1
(43) Pub. Date: Aug. 26, 2010

(54) Shear Gels and Compositions Comprising Shear Gels

(75) Inventors: Teanoosh Moaddel, Watertown, CT (US); Brian John Dobkowski, Milford, CT (US); Gabriela Maria Wis, Cos Cob, CT (US); Hasiba Pehratovic, New Britain, CT (US)

Correspondence Address: UNILEVER PATENT GROUP, 800 SYLVAN AVENUE, AG West S. Wing, ENGLEWOOD CLIFFS, NJ, 07632-3100, US

(73) Assignee: CONOPCO, INC., D/B/A UNILEVER, Englewood Cliffs, NJ (US)

(21) Appl. No.: 12/392,646

(22) Filed: Feb. 25, 2009

(51) Int. Cl. A61K 8/73; A61K 8/04

(52) U.S. Cl. 424/401; 514/54; 424/59; 424/62

(57) ABSTRACT

The present invention is directed to shear gels and topical compositions comprising the same. The topical compositions can be substantially free of silicone elastomers and yield excellent sensory characteristics when applied.

### FIELD OF THE INVENTION

The present invention is directed to shear gels and compositions comprising the same. More particularly, the present invention is directed to shear gels prepared from biopolymer particle. The shear gels can be used in topical compositions as a silicone elastomer mimetic and, when used, they unexpectedly yield topical compositions that result in excellent sensory characteristics upon application.

### BACKGROUND OF THE INVENTION

Dry skin is a problem in varying degree for most consumers. This condition is particularly evident in winter. Personal care products, such as skin creams, shampoos, shower gels and antiperspirants, are normally formulated with materials so that dry skin symptoms may be addressed.

Other consumers are concerned with the evenness of color of their skin, and particularly, age spots, freckles, as well as other blemishes resulting from hyperpigmentation. To meet the needs of consumers, a variety of attempts have been made to develop products that reduce pigment production in melanocytes.

Still other consumers are concerned with oily skin, acne and/or pores within the skin. These conditions have also been addressed in topical compositions made commercially available.

Notwithstanding the breakthroughs in treating skin conditions with topical compositions, the products developed thus far often tend to have poor sensory benefits and do not always employ desirable natural ingredients.

The present invention, therefore, is directed to shear gels and compositions comprising the same. The shear gels comprise swollen biopolymer particle (i.e., biopolymer), and are suitable for use in topical compositions as a silicone elastomer mimetic. When employed, the shear gels of the present invention, unexpectedly, result in topical compositions yielding excellent sensory characteristics upon application, and especially, an excellent silky feel when compared to conventional silicone elastomer containing products. Moreover, the shear gels of the present invention are suitable to act as silicone elastomer mimetics that are compatible with skin care actives, and compositions comprising the same have sensory characteristics similar to compositions comprising an about equal amount of silicone elastomer.

### ADDITIONAL INFORMATION

Efforts have been disclosed for making personal care products. In U.S. Patent Application Publication No. 2006/0088495A1, a personal care moistening product with dihydroxypropyltri(C₁-C₃ alkyl) ammonium salt is described.

Other efforts have been disclosed for making personal care products. In U.S. Pat. Nos. 6,875,425, 7,250,158, 7,247,294 and 7,720,805, skin lightening products are described.

Still other efforts have been disclosed that address gelling agents. In U.S. Pat. No. 5,738,897, gelled biopolymers made to impart fat-like character in consumer products are described. In European Patent Application 0432835 A1, fluid compositions with chemically setting gelling agents are described.

None of the additional information above describes shear gels prepared from biopolymer particles whereby the shear gels are suitable for use in topical compositions as a silicone elastomer mimetic.

### SUMMARY OF THE INVENTION

In a first aspect, the present invention is directed to a shear gel having been prepared with:

(a) from about 0.01 to about 15% by weight biopolymer particle; and

(b) water, polar solvent, or both

wherein the biopolymer particle is swollen to yield a biopolymer that has a swollen diameter in the shear gel that is from about 1 to about 200 microns and has been cooled with shear through its gellation temperature, the shear gel being one which is suitable for use as a silicone elastomer mimetic in a topical composition.

In a second aspect, the present invention is directed to a method for making the shear gel of the first aspect of this invention.

In a third aspect, the present invention is directed to a topical composition comprising the shear gel of the first aspect of this invention.

Topical composition, as used herein, is meant to include a composition for application to skin of mammals, especially humans. Such a composition may be generally classified as leave-on or rinse off, and is meant to include conditioners or tonics, lipsticks, color cosmetics, and general topical compositions that in some fashion and at the very least, improve a skin characteristic. Preferably, the topical composition of this invention is a leave-on composition. Lightening, as used herein, is meant to mean the lightening or whitening of skin directly as well as the lightening or whitening of spots on the skin, like age spots or freckles. Moisturizing, as used herein, means to make soft, supple and/or making the skin visually pleasing.

Comprising, as used herein, is meant to include consisting essentially of, and consisting of: Substantially free of means less than about 0.5 percent by weight, and preferably, less than about 0.3% by weight, and most preferably, from 0 to less than about 0.1% by weight based on total weight of the topical composition and including all ranges subsumed therein.

The topical composition of the present invention can, for example, be in the form of a foam, liquid, lotion, cream, serum, gel, soap bar, cleansing product (e.g., body wash, facial wash or shampoo and conditioner) or toner, or applied via a face mask or patch. Again, the topical composition of this invention is, preferably, a leave-on composition. Skin is meant to include skin on the face, neck, chest, back, arms, hands, buttocks, legs and scalp. All ranges identified herein are meant to implicitly include all ranges subsumed therein, if, for example, reference to the same is not explicitly made. Biopolymer particle, as used herein, means a macromolecule suitable for swelling with water, polar solvent or both and that may be synthetically made but is normally produced by living organisms. The biopolymer particle of the present invention can be, for example, grain-like, powdery, crystalline or the like (including freeze, and/or spray dried as described in U.S. Pat. No. 5,738,897) and is meant to mean the material supplied as a precursor to the shear gel. Swollen diameter, as used herein, means the diameter of the biopolymer particle after the same has been saturated with a swelling agent like water, a polar solvent, or both. Biopolymer, as used herein, means swollen biopolymer particle. Swollen diameter is measured by taking the longest distance between two points on the swollen particle. Swollen diameters may be determined by, for example, analyzing the diluted shear gel with an optical microscope (typically 25× magnification) under phase contrast or with a size analyzer such as a Malvern Mastersizer. Silicone elastomer mimetic means a shear gel able to mimic a silicone elastomer paste that is conventionally added to a topically applied consumer product. An example of such a silicone elastomer paste is Dow Corning® DC9045. Shear gel, as used herein, means a gel with biopolymer whereby the biopolymer has a swollen diameter that is from about 1 micron to about 200 microns, the shear gel being suitable for use as a silicone elastomer mimetic. Swollen diameter is determined by diluting shear gel with the swelling agent it is made with (about 1 part by weight shear gel to about 10 parts by weight swelling agent).

### DETAILED DESCRIPTION OF THE PREFERRED EMBODIMENTS

The only limitation with respect to the biopolymer that may be used in this invention is that the same is one which may be used in a topical composition suitable for application on to human skin. Illustrative yet non-limiting examples of the type of biopolymer suitable for use in this invention include the group consisting of polysaccharides, proteins as well as mixtures thereof.

Preferably, the biopolymer can be prepared from, for example, particles of carrageenan, furcellaran, pectin, alginate, agar, agarose, gellan, glucomannan (e.g., Konjac), galactomannan (e.g., locust bean gum, guar), xanthan, modified cellulose, glucan (e.g., starches, curdlan), gelatin, whey protein or mixtures thereof. More preferably, the biopolymer particle used is agar, agarose, carrageenan, or a mixture thereof. In a most preferred embodiment, the biopolymer particle used is agarose.

The biopolymer particles suitable for use in this invention are made commercially available from suppliers like FMC Corporation; National Starch and Chemical Co.; Cyber Colloids Ltd., as well as Hispangar, S.A. Additional descriptions of the types of biopolymer particles that may be used in this invention may be found in Food Gels, Chapter 1, edited by Peter Harris, Elsevier, 1990 and U.S. Pat. Nos. 6,673,371 and 5,738,897, the disclosures of which are incorporated herein by reference.

In an especially preferred embodiment, the biopolymer used herein is also used in conjunction with a synthetic thickener. Illustrative examples of the type of synthetic thickeners suitable for use in the shear gel of this invention include alkylated polyvinylpyrrolidones like butylated polyvinylpyrrolidone sold under the Ganex line by ISP Corporation; terephthalate polyesters like polypropylene terephthalate and ammonium acryloyldimethyltaurate/VP Copolymer, both sold under Aristoflex line by Clariant A.G.; and mono alkyl esters of poly(methyl vinyl/ether maleic acid) sodium salt, like that included in the EZ Sperse line made available by ISP Corporation, as well as (3-dimethylaminopropyl)-methacrylamide/3-methacryloylamidopropyl)-lauryl-dimethyl-ammonium chloride like that included in the Styleze line made available by ISP Corporation. Other thickeners suitable for use include those generally classified as acrylic acid/ethyl acrylate copolymers and carboxyvinyl polymers made available by the B.F. Goodrich Company under the Carbopol name. Such thickeners consist essentially of colloidally water-soluble poly-alkenyl polyether cross-linked polymer of acrylic acid crosslinked with a crosslinking agent like polyallyl sucrose or polyallyl pentaerythritol. These thickeners include, for example, Carbopol 934, 940, 950, 951, 980 and 981.

Other examples of suitable synthetic thickeners for use herein include those sold under the name Carbopol Ultrez 10, Carbopol Ultrez 21, Carbopol ETD2020, Carbopol 1342, Carbopol 1382, and Pemulen TR-1 (CTFA designation: Acrylates/10-30 Alkyl Acrylate Cross-polymer). Still other examples of suitable thickeners include those made available by Seppic under the names Sepigel 305 and Sepiplus. If desired, combinations of synthetic thickeners may be employed whereby those classified as acrylate-derived and/or terephthalate polyesters are generally preferred.

When making the shear gel of the present invention, typically from about 0.01 to about 15%, and preferably, from about 0.1 to about 10%, and most preferably, from about 0.2 to about 7% by weight biopolymer particle is used, based on total weight of the shear gel and including all ranges subsumed therein. When synthetic polymer is desired, the same typically makes up from about 0.001 to about 6%, and preferably, from about 0.01 to about 4.0%, and most preferably, from about 0.015 to about 2.5% by weight of the topical composition, based on total weight of the topical composition and including all ranges subsumed therein.

Often, the topical composition comprising the shear gel of this invention can comprise shear gel and synthetic thickener at a weight ratio from about 55 to 1 to about 1 to 55, and preferably, from about 15 to 1 to about 1 to 15, including all ratios subsumed therein. In an especially preferred embodiment, the topical composition preferably comprises (by weight) at least about 1.5 to about 8 times more shear gel than synthetic polymer.

The biopolymer typically has a swollen diameter in the shear gel from about 1 to about 200 microns, and preferably, from about 4 to about 150 microns, and most preferably, from about 8 to about 90 microns, including all ranges subsumed therein.

When making the shear gel of the present invention, biopolymer particle and water or polar solvent or both are combined and preferably mixed. The resulting biopolymer particle mixture is typically heated to a temperature that is between the gellation temperature of the resulting biopolymer and about twenty (20) percent above the melting temperature of the biopolymer. Preferably, the mixture is heated to a temperature from about 35° C. to about 100° C., and most preferably, to a temperature from about 60° C. to about 90° C., including all ranges subsumed therein.

Heating should occur for as long as it takes to yield a homogeneous mixture deplete of non-swollen biopolymer particulate. The homogeneous mixture may then be cooled with shear and through the gellation temperature of the biopolymer. Shear preferably continues until the resulting shear gel reaches ambient temperature. Shear may be provided via any art recognized technique, and preferably, under atmospheric pressure unless homogenization is desired. Apparatus/mixers typically preferred for providing shear include those made commercially available by Suppliers like ESCO-Labor AG, Ross, Plough-Share, Sonic, BEE International, Microfluidics, Arde Barinco, Inc., Serendip A.G. or Chemy Burrell. In a most preferred embodiment the shear gel of this invention is mixed with, for example, a mixer (like a u-mixer) and subsequently subjected to homogenization.

Shearing may be achieved by mixing with apparatus comprising blades, paddles or the like. Homogenization is performed, preferably, on a machine operating with a flow rate of from about 0.5 to about 2.5, and preferably, from about 0.5 to about 1.5 L/minute, including all ranges subsumed therein. The homogenizer operating pressure should be from about 1 to about 45,000 psi, preferably from about 10 to about 20,000 psi, including all ranges subsumed therein. Homogenization back pressure is typically less than 8,000 psi, and preferably, from about 0.001 to about 6000 psi, including all ranges subsumed therein. Typically the homogenization nozzle is from about 0.004 to about 0.01, and preferably, from about 0.006 to about 0.009 inches, including all ranges subsumed therein.

In another embodiment, the shear gel of the present invention may be made from a precursor block or solid specimen of gel that has been for example, cut, chopped and/or grated to yield portions or flakes of solid gel having a volume of less than about 6 mm³, and preferably, less than about 1 mm³. The portions or flakes of solid gel may be diluted with water and/or polar solvent such that the resulting mixture comprises from about 0.1 to about 15% by weight biopolymer (i.e., swollen and solid). Such a mixture may then be heated to a temperature from about 45° to 65° C. with shear and cooled, with shear, to about ambient temperature in order to produce the shear gel of this invention. In a preferred embodiment, the shear gel of this invention and originating from block or solid specimen precursor is the result of heating and shearing in a homogenizer.

The polar solvent which may be used in this invention is limited only to the extent that it can be used with biopolymer particle to make a shear gel and is hydrophilic. Illustrative yet non-limiting examples of the type of polar solvent that may be used (with or without water) in this invention are sorbitol, hydroxypropyl sorbitol, glycerine, ethoxylated glycerol, propolylated glycerol, polyalkylene glycols like polyethylene glycol and polypropylene glycol, diethylene glycol, dipropylene glycol, triethylene glycol, 2-ethoxyethanol, hexylene glycol, butylene glycol, hexamatriol, mixtures thereof or the like.

The shear gels of the present invention can make up from about 1 to about 99%, and preferably, from about 3 to about 85%, and most preferably, from about 8 to about 60% by weight of the topical composition, based on total weight of the topical composition and including all ranges subsumed therein.

It should be known, however, that commercially acceptable and conventional vehicles may be used, acting as diluents, dispersants and/or carriers for the topical compositions of this invention, along with the shear gels. Therefore, the cosmetically acceptable vehicle suitable for use in this invention may be aqueous-based, anhydrous, oil-based or an emulsion, including a multiple emulsion. If the use of water is desired as a carrier, water typically makes up the balance of the topical composition. Silicone elastomers are typically not preferred in this invention since the shear gels described herein are, surprisingly, excellent silicone elastomer mimetics. Optionally, however, silicone elastomers may be used along with the shear gels.

In addition to water, organic solvents may be optionally included to act as carriers along with the shear gels within the topical compositions of the present invention. Illustrative and non-limiting examples of the types of organic solvents suitable for use in the present invention include alkanols like ethyl and isopropyl alcohol, mixtures thereof or the like.

Other optional additives suitable for use as carriers along with the shear gels of this invention include ester oils like isopropyl myristate, cetyl myristate, 2-octyldodecyl myristate, avocado oil, almond oil, olive oil, sunflower seed oil, neopentylglycol dicaprate, mixtures thereof or the like. Typically, such ester oils are used at an amount to yield a stable, and most preferably, water-in-oil emulsion when such an emulsion is desired. Other oils suitable for use include those generally classified as hydrocarbons, including those known as waxes, and jellies, including petroleum jelly.

Emollients may also be used, if desired, as carriers within the topical composition of the present invention. Alcohols like 1-hexadecanol (i.e., cetyl alcohol) are often desired as are the emollients generally classified as silicone oils and synthetic esters. Silicone oils suitable for use include cyclic or linear polydimethylsiloxanes containing from 3 to 9, preferably from 4 to 5, silicon atoms. Nonvolatile silicone oils useful as an emollient material in the inventive topical composition described herein include polyalkyl siloxanes, polyalkylaryl siloxanes and polyether siloxane copolymers. The essentially non-volatile polyalkyl siloxanes useful herein include, for example, polydimethylsiloxanes.

The ester emollients that may optionally be used are:
(1) Alkenyl or alkyl esters of fatty acids having 10 to 20 carbon atoms. Examples thereof include isoarachidyl neopentanoate, isononyl isonanonoate, oleyl myristate, oleyl stearate, and oleyl oleate.
(2) Ether-esters such as fatty acid esters of ethoxylated fatty alcohols.
(3) Polyhydric alcohol esters. Ethylene glycol mono- and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters.
(4) Wax esters such as beeswax, spermaceti, stearyl stearate and arachidyl behenate.
(5) Sterol esters, of which cholesterol fatty acid esters are examples.

Emollients, when used, typically make up from about 0.1 to about 50% by weight of the topical composition, including all ranges subsumed therein.

Fatty acids having from 10 to 30 carbon atoms may also be included as cosmetically acceptable carriers within the composition of the present invention. Illustrative examples of such fatty acids include pelargonic, lauric, myristic, palmitic, stearic, isostearic, oleic, linoleic, arachidic, behenic or erucic acid, and mixtures thereof. Compounds that are believed to enhance skin penetration, like dimethyl sulfoxide, may also be used as an optional carrier.

The polar solvents described herein may also be added as humectants in the desired topical composition of this invention. Therefore, such polar solvents may be used to make shear gel, only as a humectant as an additive to the topical composition, or both. In an especially preferred embodiment, the topical composition of this invention has less than about 50% by weight polar solvent, and preferably, from about 0.001 to about 25% by weight polar solvent based on total weight of the topical composition and including all ranges subsumed therein.

Collectively, water, shear gels, silicones, esters, fatty acids and/or humectants will constitute the cosmetically acceptable carrier in amounts from 1 to 99.9%, preferably from 80 to 99% by weight.

Surfactants may also be present in the topical compositions of the present invention. Total concentration of the surfactant will range from about 0 to about 40%, and preferably, from about 0 to about 20%, optimally from about 0.001 to about 5% by weight of the composition. The surfactant may be selected from the group consisting of anionic, nonionic, cationic and amphoteric actives. Particularly preferred nonionic surfactants are those with a C₁₀-C₂₀ fatty alcohol or acid hydrophobe condensed with from 2 to 100 moles of ethylene oxide or propylene oxide per mole of hydrophobe; mono- and di-fatty acid esters of ethylene glycol; fatty acid monoglyceride; sorbitan, mono- and di-C₈-C₂₀ fatty acids; block copolymers (ethylene oxide/propylene oxide); and polyoxyethylene sorbitan as well as combinations thereof. Alkyl polyglycosides and saccharide fatty amides (e.g. methyl gluconamides) are also suitable nonionic surfactants.

Preferred anionic surfactants include soap, alkyl ether sulfate and sulfonates, alkyl sulfates and sulfonates, alkylbenzene sulfonates, alkyl and dialkyl sulfosuccinates, C₈-C₂₀ acyl isethionates, acyl glutamates, C₈-C₂₀ alkyl ether phosphates and combinations thereof. In an especially preferred embodiment, the surfactant employed is nonionic, and especially, polyoxyethylene sorbitan monopalmitate sold as Tween 40 by ICI Americas, Inc.

Perfumes may be used in the topical composition of this invention. Illustrative non-limiting examples of the types of perfumes that may be used include those comprising terpenes and terpene derivatives like those described in Bauer, K., et al., Common Fragrance and Flavor Materials, VCH Publishers (1990).

Illustrative yet non-limiting examples of the types of fragrances that may be used in this invention include myrcene, dihydromyrenol, citral, tagetone, cis-geranic acid, citronellic acid, mixtures thereof or the like.

Preferably, the amount of fragrance employed in the topical composition of this invention is in the range from about 0.0% to about 10%, more preferably, about 0.00001% to about 5 wt %, most preferably, about 0.0001% to about 2%.

Various types of optional ingredients/additives may be used in the topical compositions of the present invention. Although not limited to this category, general examples include talcs and silicas, as well as alpha-hydroxy acids, beta-hydroxy acids, zinc salts, and sunscreens.

Beta-hydroxy acids include salicylic acid, for example. Zinc pyrithione is an example of the zinc salts useful in the topical composition of the present invention.

Sunscreens include those materials commonly employed to block ultraviolet light. Illustrative compounds are the derivatives of PABA, cinnamate and salicylate. For example, avobenzophenone (Parsol 1789®) octyl methoxycinnamate and 2-hydroxy-4-methoxy benzophenone (also known as oxybenzone) can be used. Octyl methoxycinnamate and 2-hydroxy-4-methoxy benzophenone are commercially available under the trademarks, Parsol MCX and Benzophenone-3, respectively. The exact amount of sunscreen employed in the compositions can vary depending upon the degree of protection desired from the sun's UV radiation. Additives that reflect or scatter the suns rays may also be employed. These additives include oxides like zinc oxide and titanium dioxide.

Many topical compositions, especially those containing water, should be protected against the growth of potentially harmful microorganisms. Anti-microbial compounds, such as triclosan, and preservatives are, therefore, typically necessary. Suitable preservatives include alkyl esters of p-hydroxybenzoic acid, hydantoin derivatives, propionate salts, and a variety of quaternary ammonium compounds. Particularly preferred preservatives of this invention are methyl paraben, propyl paraben, phenoxyethanol and benzyl alcohol. Preservatives will usually be employed in amounts ranging from about 0.1% to 2% by weight of the topical composition.

Still other optional ingredients/additives that may be used in the topical composition of this invention include chelators like EDTA, pH modifiers (e.g., NaOH), dioic acids (e.g., malonic acid, sebacic acid), antioxidants like vitamin E, retinoids, including retinoic acid, retinal, retinol and retinyl esters, conjugated linoleic acid, petroselinic acid and mixtures thereof, as well as any other conventional ingredients well known for wrinkle-reducing, anti-acne effects and reducing the impact of sebum.

Even other optional additives that may be employed in the topical composition of the present invention are skin lightening additives. Illustrative yet non-limiting examples of skin lightening additives that may be used in this invention are niacinamide, vitamin C and its derivatives, 12-hydroxystearic acid, resorcinols and their derivatives (including those esterified with, for example, ferulic acid, vanillic acid or the like), extracts of kudzu, chamomile, and yarrow as well as any mixtures of the skin lightening sources.

Often preferred optional additives suitable for use in the topical composition of this invention include sensory modifying particles like microcrystalline cellulose, silica modified ethylene/methacrylate copolymer microspheres, talc modified ethylene/methacrylate copolymer microspheres, mixtures thereof or the like. Other examples of the types of particles suitable for use in this invention include those comprising polyolefins like polyethylene, polypropylene and/or polybutylene-based polymers, polyamides (like nylon fibers), mixtures thereof or the like. Still other preferred particles suitable for use in this invention include those comprising polyurethane, polystyrene, epoxy resins, urea resins, silicone resins, mixtures thereof or the like.

In a preferred embodiment, the particles used in this invention comprise polyethylenes, or are talc comprising particles or mixtures thereof. The former are often sold under the names Cerapure (made commercially available by Shamrock), Asensa (made commercially available by Honeywell) and Miperon (made commercially available by Mitsui Petrochemical Industries, Ltd.). Another preferred polyethylene-based particle is sold under the name CL-2080 (made commercially available by Kobo Industries). Other preferred particles suitable for use in this invention include nylons (e.g., nylon-12) sold under the name SP-10 which is made commercially available by Kobo Industries. Still other preferred particles suitable for use in this invention include those comprising copolymers of ethylene and methacrylate that contain silica or talc and sold under the names SPCAT-12 and DSPCS-12, respectively, both of which are also made commercially available by Kobo Industries. Other particles comprising polystyrenes and polymethyl methacrylate (sold, for example, under the names Ganzpearl GS-0605 and GME0380, respectively) and made available from Presperse are also often preferred.

Even other particles suitable for use in this invention include natural polymeric spheroids like those which comprise starch and those which comprise silk, the former, for example, made available from National Starch and Chemical and the latter, for example, made available by Engelhard Corporation. Still other natural polymeric particles suitable for use in this invention include those natural polymeric particles comprising cellulose such as Celluflow and Cellulo Beads, the former made commercially available by Chisso Corporation and the latter made available by Kobo Industries.

When used, such particles typically make up from about 0.001 to about 10%, and preferably, from about 0.01 to about 8%, and most preferably, from about 0.1 to about 6% by weight of the total weight of the topical composition, including all ranges subsumed therein.

Other preferred optional additives suitable for use with the shear gels of this invention include moisturizing agents like hydroxypropyl tri(C₁-C₃ alkyl)ammonium salts. These salts may be obtained in a variety of synthetic procedures, most particularly by hydrolysis of chlorohydroxypropyl tri(C₁-C₃ alkyl)ammonium salts. A most preferred species is 1,2-dihydroxypropyltrimonium chloride, wherein the C₁-C₃ alkyl is a methyl group. Amounts of the salt may range from about 0.2 to about 30%, and preferably from about 0.5 to about 20%, optimally from about 1% to about 12% by weight of the topical composition, including all ranges subsumed therein.

Ordinarily the C₁-C₃ alkyl constituent on the quaternized ammonium group will be methyl, ethyl, n-propyl, isopropyl or hydroxyethyl and mixtures thereof. Particularly preferred is a trimethyl ammonium group known through INCI nomenclature as a “trimonium” group. Any anion can be used in the quat salt. The anion may be organic or inorganic with proviso that the material is cosmetically acceptable. Typical inorganic anions are halides, sulfates, phosphates, nitrates and borates. Most preferred are the halides, especially chloride. Organic anionic counter ions include methosulfate, toluoyl sulfate, acetate, citrate, tartrate, lactate, gluconate, and benzenesulfonate.

Still other preferred moisturizing agents which may be used, especially in conjunction with the aforementioned ammonium salts include substituted urea like hydroxymethyl urea, hydroxyethyl urea, hydroxypropyl urea; bis(hydroxymethyl) urea; bis(hydroxyethyl) urea; bis(hydroxypropyl) urea; N,N′-dihydroxymethyl urea; N,N′-di-hydroxyethyl urea; N,N′-di-hydroxypropyl urea; N,N,N′-tri-hydroxyethyl urea; tetra(hydroxymethyl) urea; tetra(hydroxyethyl) urea; tetra(hydroxypropyl urea; N-methyl, N′-hydroxyethyl urea; N-ethyl-N′-hydroxyethyl urea; N-hydroxypropyl-N′-hydroxyethyl urea and N,N′dimethyl-N-hydroxyethyl urea. Where the term hydroypropyl appears, the meaning is generic for either 3-hydroxy-n-propyl, 2-hydroxy-n-propyl, 3-hydroxy-1-propyl or 2-hydroxy-1-propyl radicals. Most preferred is hydroxyethyl urea. The latter is available as a 50% aqueous liquid from the National Starch & Chemical Division of ICI under the trademark Hydrovance.

Amounts of substituted urea that may be used in the topical composition of this invention range from about 0.01 to about 20%, and preferably, from about 0.5 to about 15%, and most preferably, from about 2 to about 10% based on total weight of the composition and including all ranges subsumed therein.

When ammonium salt and substituted urea are used, in a most especially preferred embodiment at least from about 0.01 to about 25%, and preferably, from about 0.2 to about 20%, and most preferably, from about 1 to about 15% humectant, like glycerine, is used, based on total weight of the topical composition and including all ranges subsumed therein. In yet another especially preferred embodiment, the topical composition of this invention is substantially free of silicone elastomer.

The topical composition of the present invention is intended for use primarily as a product for topical application to human skin, especially and at least as a product that may moisturize the skin. Thus, the inventors have discovered that the described shear gels unexpectedly can be used as an excellent base in a topical composition to deliver excellent sensory benefits (e.g., silkiness) when the topical composition is, for example, substantially free of silicone elastomer. Other benefits from using the topical composition of this invention may include skin lightening, decreasing the effect of sebum on the skin and skin wrinkle reducing. In an especially preferred embodiment, the topical composition of the present invention has a pH from about 4.5 to about 7.5, including all ranges subsumed therein. In yet another especially preferred embodiment, the shear gel of the present invention predominately carries additives and, therefore, is not required to entrap or encapsulate additive. Moreover, the shear gel of the present invention typically has a viscosity from about 4,000 to about 30,000, and preferably, from about 8,000 to about 25,000, and most preferably, from about 12,000 to about 23,000 cps initially and after 24 hours at ambient temperature (measured with a Brookfield DV-1 Viscometer, with RV-S06 spindle, 25° C., 20 rpm).

When making the topical composition of the present invention, the desired shear gel and other ingredients are mixed in no particular order and usually at temperatures from about 50 to about 80° C. and under atmospheric pressure.

The packaging for the composition of this invention can be a patch, bottle, tube, roll-ball applicator, propellant driven aerosol device, squeeze container or lidded jar.

The examples which follow are provided to illustrate and facilitate an understanding of the invention. The examples are not intended to limit the scope of the claims.

### Example 1

2% agarose shear gels were made by mixing, with shear, agarose and water in a manner consistent with the process steps of this invention. The apparatus used was a U-mixer made commercially available by Arde Barinco, Inc.

3% agarose shear gels were also made by mixing agarose, with shear provided by a u-mixer, in a manner consistent with the process steps of this invention.

The shear gels in this example were further processed with a homogenizer supplied by Bee International and consistent with the processing steps described herein.

The following ingredients were combined to make a topical compositions consistent with this invention. The resulting topical compositions were mixed, homogenized or both in order to produce a homogeneous end use product with biopolymer having swollen diameter consistent with this invention. Synthetic thickener was neutralized with base either before or after addition.

| | Topical | Topical | Topical |
|---|---|---|---|
| | Composition I | Composition II | Composition III |
| Ingredient | % by Weight | % by Weight | % by Weight |
| Water | Balance | Balance | Balance |
| Synthetic thickener | 1.0 | 1.0 | 1.5 |
| (Carbopol | | | |
| Ultrez 21) | | | |
| Nonionic surfactant | 0.7 | 0.7 | 1.2 |
| Humectant | 15.0 | 15.0 | 15 |
| Preservative | 0.7 | 0.7 | 0.7 |
| Shear gel | 15.0* | 10.0*** | — |
| NaOH (50%) | 0.6 | 0.5 | 0.5 |
| Moisturizing agent | 10 | 10 | 10 |
| Silicone oil | 9.0 | 9.0 | 9.0 |
| Mineral oil | 1.0 | 1.0 | 1.0 |
| Sensory modifying | 2.0** | 2.0** | 2.0**** |
| particle | | | |
| Chelator | — | — | 0.05 |
| Fragrance | 0.3 | 0.3 | 0.3 |

*2% agarose shear gel, water solvent
**polymethylmethacrylate (KOBO, ES-830)
***3% agarose shear gel, water solvent
****methylmethacrylate crosspolymer (KOBO, MSP-825)

### Example 2

The topical compositions of Example 1 were evaluated by panelists. All panelists applied about 0.1 mL of composition onto about a 38 cm² section of their volar forearm and assessed the same for absorption speed and three skin feels (greasiness, stickiness and silkiness) immediately after application and after about five (5) minutes of absorption. All panelists concluded that topical compositions made according to this invention were overall perceived as less sticky and greasy than the compositions made free of shear gel. The panelists also concluded that the compositions made according to this invention were perceived to absorb faster into the skin than compositions free of shear gel. Moreover, all panelists concluded that the compositions of the present invention were perceived as more silky overall than compositions deplete of the shear gel of this invention.

### Example 3

The following ingredients were combined to make topical compositions consistent with this invention.

| Ingredients | % by Weight |
|---|---|
| Water | Balance |
| Synthetic thickener (Aristoflex AVC) | 0.5 |
| Chelator | 0.050 |
| Anionic surfactant | 1.2 |
| Humectant | 3.5 |
| Preservative | 0.7 |
| 3% agarose sheer gel | 10.0 |
| Silicone oil | 41.0 |
| Mineral oil | 1.5 |
| Sensory modifying particle | 4.000 |
| Fragrance | 0.300 |

The topical compositions of this example were compared to a commercially available Pond's Fine Pore product containing 40% silicone elastomer blend and no shear gel. A bilateral comparison was made by panelists and all panelists surprisingly concluded that the composition made with shear gel and consistent with this invention was, after application, comparable in silkiness to a superior commercial product containing 40% silicone elastomer blend.

### Example 4

The following ingredients were combined to make topical compositions consistent with this invention and with 40% by weight shear gel. Shear gel and topical compositions were made in a manner similar to the one described in Example 1.

| Ingredient | wt % |
|---|---|
| Water | Balance |
| Humectant | 12.000 |
| Preservative | 0.350 |
| Surfactant | 2.000 |
| Synthetic Thickener (Aristoflex AVC) | 0.750 |
| 4% Agarose Shear Gel | 40.000 |

The topical compositions of this example were applied by panelists. All concluded that the composition delivered a silky skin feel and were not sticky, notwithstanding the fact that 40% by weight shear gel was used.

What is claimed:

1. A shear gel having been prepared from:
(a) from about 0.01 to about 15% by weight biopolymer; and
(b) water, polar solvent or both
wherein the biopolymer particle is swollen to yield a biopolymer that has a swollen diameter in the shear gel that is from about 1 to about 200 microns and has been cooled with shear through its gellation temperature, the shear gel being one which is suitable for use as a silicone elastomer mimetic in a topical composition.

2. The shear gel according to claim 1 wherein the biopolymer comprises carrageenan, furcellaran, pectin, alginate, agar or agarose, gellan, glucomannan, galactomannan, xanthan, modified cellulose, glucan, gelatin, whey protein or a mixture thereof.

3. The shear gel according to claim 1 wherein the biopolymer comprises agar or agarose.

4. The shear gel according to claim 1 wherein the biopolymer has a swollen diameter in the shear gel from about 8 to about 150 microns.

5. The shear gel according to claim 1 wherein the biopolymer is swollen with water.

6. The shear gel according to claim 1 wherein the biopolymer is swollen with polar solvent.

7. A topical composition comprising:
(i) a shear gel having been prepared with:
(a) from about 0.01 to about 12% by weight biopolymer; and
(b) water, polar solvent or both; and
(ii) additive
wherein the biopolymer particle is swollen to yield a biopolymer that has a swollen diameter in the shear gel that is from about 1 to about 200 microns and has been cooled with shear through its gellation temperature, the shear gel being one which is suitable for use as a silicone elastomer mimetic in a topical composition.

8. The topical composition according to claim 7 wherein the topical composition further comprises cosmetically acceptable carrier.

9. The topical composition according to claim 7 wherein the biopolymer comprises carrageenan, furcellaran, pectin, alginate, agar or agarose, gellan, glucomannan, galactomannan, xanthan, modified cellulose, glucan, gelatin, whey protein or a mixture thereof.

10. The topical composition according to claim 7 wherein the biopolymer particle comprises agar or agarose.

11. The topical composition according to claim 7 wherein the biopolymer has a swollen diameter in the shear gel from about 8 to about 150 microns.

12. The topical composition according to claim 7 wherein the biopolymer is swollen with water.

13. The topical composition according to claim 7 wherein the biopolymer is swollen with polar solvent.

14. The topical composition according to claim 6 wherein the polar solvent comprises sorbitol, hydroxypropyl sorbitol, glycerol, ethoxylated glycerol, propolylated glycerol, polyalkylene glycols like polyethylene glycol and polypropylene glycol, diethylene glycol, dipropylene glycol, triethylene glycol, 2-ethoxyethanol, hexylene glycol, butylene glycol, hexamatriol or a mixture thereof.

15. The topical composition according to claim 7 wherein the shear gel makes up from about 1 to about 99% by weight of the topical composition.

16. The topical composition according to claim 7 wherein the topical composition is substantially free of silicone elastomer.

17. The topical composition according to claim 7 wherein the cosmetically acceptable carrier comprises dimethicone.

18. The topical composition according to claim 7 wherein the additive comprises a skin lightening additive, a wrinkle-reducing agent, a sunscreen, a vitamin or a moisturizing agent.

19. The topical composition according to claim 7 wherein the additive comprises hydroxypropyl tri(C₁-C₃ alkyl) ammonium salt, a substituted urea, glycerine or a mixture thereof.

20. The topical composition according to claim 7 wherein the topical composition further comprises a surfactant, sensory modifying particle, synthetic thickener or a mixture thereof.

21. A method for making a shear gel comprising the steps of:
(a) combining and mixing biopolymer, and water or polar solvent or both;
(b) heating the resulting biopolymer mixture to a temperature that is between the gellation temperature of the biopolymer and about twenty percent above the melting temperature of the biopolymer to produce a homogeneous mixture deplete of non-swollen biopolymer particulate;
(c) cooling the homogeneous mixture with shear and through the gellation temperature of the biopolymer until a shear gel at about ambient temperature is recovered.

22. A method for making a shear gel comprising the steps of:
(a) chopping, cutting and/or grating a precursor gel block or specimen to yield gel portions or flakes having a volume of less than about 6 mm³;
(b) diluting the portions or flakes with water and/or polar solvent to produce a mixture;
(c) heating the mixture to a temperature from about 45° to about 65° C. with shear; and
(d) cooling the heated mixture, with shear, to about ambient temperature to produce the shear gel.
